(19) United States
(12) Patent Application Publication
(10) Pub. No.: US 2023/0142625 A1
(43) Pub. Date: May 11, 2023

(54) METHOD AND SYSTEM OF PROVIDING PERSONALIZED GUIDELINE INFORMATION FOR A USER IN A PREDETERMINED DOMAIN

(71) Applicant: NEC Laboratories Europe GmbH, Heidelberg (DE)

(72) Inventors: Carolin LAWRENCE, Heidelberg (DE); Timo SZTYLER, Heidelberg (DE)

(21) Appl. No.: 17/919,755

(22) PCT Filed: Jun. 2, 2020
(86) PCT No.: PCT/EP2020/065242

§ 371 Date: Oct. 19, 2022

(51) Int. Cl. G06N 20/00; G06F 18/22; A61B 5/16

(52) U.S. Cl. CPC G06N 20/00; G06F 18/22; A61B 5/165; A61B 5/167

(57) ABSTRACT

A method of providing personalized guideline information for a user in a predetermined domain, in which a set of personality types is defined for users of said predetermined domain, includes: determining, by a personality type recognizer, a personality type for a user in order to assign the personality type to said user, selecting a personality-typed machine learning model from a model pool of personality-typed machine learning models based on the personality type of said user, where the selected personality-typed machine learning model is used to initialize an individual personalized machine learning model of said user, and generating, by the individual personalized machine learning model of said user, a recommendation prediction, the recommendation prediction is presented as a guideline information to said user.

### CROSS REFERENCE TO RELATED APPLICATIONS

This application is a U.S. National Phase application under 35 U.S.C. § 371 of International Application No. PCT/EP2020/065242, filed on Jun. 2, 2020. The International Application was published in English on Dec. 9, 2021, as WO 2021/244734 A1 under PCT Article 21(2).

### FIELD

The present invention relates to a method and system of providing personalized guideline information for a user in a predetermined domain, in particular in the context of everyday activities.

### BACKGROUND

Personal digital assistants and corresponding methods can help humans to reach their personal activity goals, such as a healthier lifestyle. With respect to the current state of the art in the context of assisting humans, for example, it is referred to the following non-patent literature:
Michie, Susan, et al. “The Human Behaviour-Change Project: harnessing the power of artificial intelligence and machine learning for evidence synthesis and interpretation.” Implementation Science 12.1 (2017): 121.
Civitarese, Gabriele, et al. “NECTAR: Knowledge-based collaborative active learning for activity recognition.” 2018 IEEE International Conference on Pervasive Computing and Communications (PerCom). IEEE, 2018.
Sztyler, Timo, and Heiner Stuckenschmidt. “Online personalization of cross-subjects based activity recognition models on wearable devices.” 2017 IEEE International Conference on Pervasive Computing and Communications (PerCom). IEEE, 2017.
McBurney, Sarah, et al. “Adapting pervasive environments through machine learning and dynamic personalization.” 2008 IEEE International Symposium on Parallel and Distributed Processing with Applications. IEEE, 2008.
Richa Khandelwal. “Serving Athletes* With Personalized Workout.” 2018, which is retrievable at https://medium.com/nikeengineering/serving-athletes-with-personalized-workout-recommendations-285491eabc3d.
Tkalcic et al. 2014. “Personality and Emotions in Decision Making and Recommender Systems.” Proceedings of the First International Workshop on Decision Making and Recommender Systems (DMRS2014), which is retrievable at http://ceur-ws.org/Vol-1278/paper3.pdf.

Document US 2016/0171514 A1 refers to systems, methods, and/or computer-readable media that enable computation of various types of crowd-based results regarding experiences users may have in their day-to-day life.

In WO 2016/105637 A1 systems and methods are disclosed for determining an affective state of a user. A user behavior characteristic is detected in response to content provided to the user. Content metadata indicates a context of the content provided to the user and a probability of the user experiencing at least one expected emotion in response to an interaction with the content. Based on the context and the at least one expected emotion indicated in the content metadata, one or more rules are applied to map the detected user behavior characteristic to an affective state of the user.

A digital assistant system or method that suggests activities to a person in order for them to reach their activity goals, needs to know how to best motivate this person. Because every person is different, one model for all users would lead to inferior results. Instead, models should be personalized. However, at the beginning, when the user uses the assistant system for the first time, there is no data available to personalize the model. Additionally, the feedback data that a user generates is scarce because it is a bandit learning setup: The system suggests an activity and feedback for this activity is obtained. At one point in time, the model can only suggest one activity and it is unknown what feedback would have been obtained for another activity. Because of this scarcity, it would be beneficial if data could be shared across users. However, this is not possible in a straight-forward manner because the users have different personalities and simply reusing another user's feedback data would lead to an ill-suited model.

### SUMMARY

In an embodiment, the present disclosure provides a method of providing personalized guideline information for a user in a predetermined domain, wherein a set of personality types is defined for users of said predetermined domain, the method comprises: determining, by a personality type recognizer, a personality type for a user in order to assign the personality type to said user, selecting a personality-typed machine learning model from a model pool of personality-typed machine learning models based on the personality type of said user, wherein the selected personality-typed machine learning model is used to initialize an individual personalized machine learning model of said user, and generating, by the individual personalized machine learning model of said user, a recommendation prediction, wherein the recommendation prediction is presented as a guideline information to said user.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic view illustrating an excerpt of the psychological types (personalities) by Myers-Briggs;

FIG. 2 is a schematic diagram illustrating a workflow for a method according to an embodiment of the present invention; and

FIG. 3 is a schematic diagram illustrating a collaborative personality-based feedback harmonizer for a method according to an embodiment of the present invention.

### DETAILED DESCRIPTION

In view of the above, the present disclosure improves and further develops a method and a system of the initially described type of providing personalized guideline information for a user in a predetermined domain in such a way that the guideline information can be provided in an improved and efficient way.

In accordance with the invention, the aforementioned is accomplished by a method of providing personalized guideline information for a user in a predetermined domain, in particular in the context of everyday activities, wherein a set of personality types is defined for users of said predetermined domain, the method comprising:

determining, by a personality type recognizer, a personality type for a user in order to assign the personality type to said user;

selecting a personality-typed machine learning model from a model pool of personality-typed machine learning models based on the personality type of said user, wherein the selected personality-typed machine learning model is used to initialize an individual personalized machine learning model of said user; and

generating, by the individual personalized machine learning model of said user, a recommendation prediction, wherein the recommendation prediction is presented as a guideline information to said user.

Furthermore, the aforementioned is accomplished by a system of providing personalized guideline information for a user in a predetermined domain, wherein a set of personality types is defined for users of said predetermined domain, the system is configured

to determine, by a personality type recognizer, a personality type for a user in order to assign the personality type to said user,

to select a personality-typed machine learning model from a model pool of personality-typed machine learning models based on the personality type of said user, wherein the selected personality-typed machine learning model is used to initialize an individual personalized machine learning model of said user, and

to generate, by the individual personalized machine learning model of said user, a recommendation prediction, wherein the recommendation prediction is presented as a guideline information to said user.

According to the invention, it has first been recognized that best activity suggestion—e.g., in the form of recommendation prediction as guideline information—may be highly dependent on the personality of the user and this should be respected by a digital assistant method or system in order to achieve the best possible activity predictions and guideline information for a user.

According to the invention, a personality type recognizer determines a personality type for a user in order to assign the personality type to the user. Then a personality-typed machine learning model is selected from a model pool of personality-typed machine learning models. This selection is based on the personality type of the user, wherein the selected personality-typed machine learning model is used to initialize an individual personalized machine learning model for the user. The individual personalized machine learning model of the user generates a recommendation prediction, wherein the recommendation prediction is presented as a guideline information to the user.

Thus, the present invention provides a method and a system of providing personalized guideline information for a user in a predetermined domain, in particular in the context of everyday activities, wherein the guideline information can be provided in an improved and efficient way.

According to embodiments of the invention, users are clustered by personality type and each user may be given an initial assistant model, i.e. a personality-typed machine learning model, trained for their assigned personality type.

The model may collect feedback from each user, which is reweighed by their mood at the time and can be stored in a shared database across all users. Using a collaborative personality-based feedback harmonizer, the data of all users can be reweighed so that it can be used to update personalized models for each user as well as the initial personality-typed machine learning models.

Thus, embodiments of the disclosure may provide the following:
They offer a good initial model by grouping users into personality types.
They offer a method with which data from all users can be used to personalize the models of other users as well as the initial personality-typed models.

The term “machine learning model” may refer to a mathematical representation of a process, which can be generated with an algorithm using training data. According to embodiments of the invention, the machine learning model may be an artificial neural network (ANN).

According to embodiments of the invention, it may be provided that the personality-typed machine learning models of the model pool were trained based on previously collected data. Furthermore, there may be one personality-typed machine learning model for each personality type.

According to embodiments of the invention, a controller module may be provided, wherein the controller module is configured to inform the individual personalized machine learning model such that model decisions, in particular recommendation predictions, can be influenced.

According to embodiments of the invention, it may be provided that every time the individual personalized machine learning model presents a recommendation prediction to the user, the individual personalized machine learning model is informed by the controller module, wherein the controller module in turn considers the recognized personality type of the user.

According to embodiments of the invention, the controller module may be configured to interact with the user. For example, the controller module may be configured to interact with the user and to inform the individual personalized machine learning model, such that a model decision, in particular a recommendation prediction, can be influenced.

According to embodiments of the invention, the control module may be configured to collect/receive feedback information from the user when a recommendation prediction is presented to the user. The feedback information may include implicit and/or explicit feedback information. For example, it may be provided that each time an action is selected and shown to the user as recommendation prediction and guideline information, implicit and explicit feedback is collected. For instance, implicit feedback may be collected by measuring the heart rate change after the treadmill speed has been increased. Explicit feedback can be collected by outright asking the user to provide feedback on whether or not they liked the change in speed. Furthermore, to allow the user to have more control, they may also provide more fine-grained feedback such as that the suggested workout time was too long or by selecting or providing a suggestion that they would have preferred. Based on this feedback, the model can adjust to be more uniquely suited for this user.

According to embodiments of the invention, the controller module may include an emotional state recognizer, wherein the emotional state recognizer is employed to handle implicit feedback information of the user such that an emotional state of the user is determined. The emotional state recognizer might for example register skin temperature, perspiration levels, heart pressure, blood pressure, voice pitch, etc. With this information the system may gauge the emotional state of the user, e.g. if the user is stressed. This information might be used in two ways: First, it can inform the predictive model so that the chosen action (as guideline information) depends on the emotional state (e.g. if the user is stressed it might be better not to lower the speed of the treadmill again). Second, it can be used to reweigh feedback given by the user (e.g. feedback given while the user is stressed could be deemed more or less valuable).

According to embodiments of the invention, it may be provided that the emotional state of the user is considered by the individual personalized learning model of the user.

According to embodiments of the invention, the emotional state of the user may be used to reweigh the feedback information given by the user.

According to embodiments of the invention, the feedback information may be logged/stored together with the corresponding recommendation prediction in a feedback database, wherein the feedback database is shared across all users of the predetermined domain.

According to embodiments of the invention, it may be provided that, e.g. periodically, feedback information of users collected by the feedback database is used to update all personality-typed machine learning models of the model pool and/or to update all users' individual personalized machine learning models.

According to embodiments of the invention, it may be provided that, if a final outcome of a user's logged feedback information is known (e.g. whether the user reached his goal or not), all feedback information data points of the user are readjusted by the final outcome in a post episode feedback adjustment.

According to embodiments of the invention, it may be provided that a user similarity matrix is computed, wherein the user similarity matrix indicates how similar each user is to all other users. Thus, based on a similarity score, logged feedback information of the feedback database may be reweighed to create a personalized log for a specific user, wherein the personalized log is used to update the individual personalized machine learning model of the specific user.

According to embodiments of the invention, it may be provided that, depending on a personality type mismatch between the personality type assigned to a user and a specific personality type of a specific personality-typed machine learning model that is to be updated, logged feedback information of the feedback database is reweighed to create a personality-typed log. The personality-typed log may then be used to update the specific personality-typed machine learning model having the specific personality type.

According to embodiments of the invention, the predetermined domain may include a gym environment, a smart home environment, a virtual reality environment, a shopping environment and/or a health environment. In this regard, further features, advantages and further embodiments are described or may be become apparent in the following:

### 1. Gym Environment:

A gym trainer needs to analyze the trainees and piece together their performance, weaknesses, needs, (emotional) state and objectives. From a practical point of view, a trainer could never keep the data from as many users in mind as a machine can. Further, they cannot handle sensor data such as blood pressure or stress-level over a longer period. Embodiments of the invention can be used to keep track of all this information, combine them, and in turn provide personality-based and personalized feedback and recommendations. This can be a personalized training plan, which is adjusted on demand. Furthermore, the output of embodiments of the invention can be used to adapt and personalize automatically the sports equipment, e.g., in respect of the weights or the speed.

### 2. Smart-Home Environment:

Embodiments of the invention can be situated in a smart-home environment with a sensor network (fridge, light, temperature, movement, object interaction, doors, sound system, etc.) which is used to monitor and support inhabitants. The home recognizes the user's actions and activities and evaluates the daily routine for further purposes (e.g. smart alarm for elderly people). In this scenario, embodiments of the invention can help to full-fill targets of the day (e.g. personalized order of the tasks to maximize the motivation), but also to automatically adapt the settings (e.g. lighting) to the user's needs. In addition, the personality-based instructions of embodiments of the invention can be used to automatically improve the performance of the sensor-network by adapting certain parameters like how to interpret the events reported by the sensor network. In this context, the output of the sensor network but also how the user reacts to certain changes within the environment can be in turn used to improve the personality-type-based machine learning model.

### 3. Virtual Reality Environment

A virtual reality is a simulated experience where users are entertained, taught, or even treated. While embodiments of the invention can be used to help the user (independent of the scenario) e.g. to maximize the happiness or satisfaction by providing recommendations what the user should or can do within the virtual environment, simultaneously the output of embodiments of the invention can be used to adapt the appearance, the sound, or plot of the virtual reality. For example, assume the user enters the virtual reality to play a computer game, then the character models and the level of difficulty can be automatically adapted to the user's preferences or (emotional) state. In turn, the users' reaction but also the one of similar users (multiplayer) to these changes within the virtual reality can be considered as feedback to continually improve the model and so the experience of the user.

### 4. Shopping Environment

Nowadays, users consider a lot of aspects beside costs, e.g. if a product is CO2 neutral, fair trade, vegan, or transparency in respect of its production. Embodiments of the invention can be embodied in a system that helps the user to maximize these aspects while shopping by providing product recommendations or automatically generating e.g. a grocery list. For instance, the user plans to cook and wants to use ingredients, which maximize the trade-off between price, fair trade, and edibility. Embodiments of the invention leads the user and also considers the feedback in respect of the result to optimize and personalize the recommendations. Moreover, the generated shopping list can be automatically forwarded to a (local) provider to order the desired items.

### 5. Health Environment

The dosage of drugs but also vitamins have to be usually adjusted or personalized on demand depending on various factors. First, embodiments of the invention can be used to guide the user and to provide recommendations based on the user's condition (e.g. considering blood test results) as well as external factors (e.g. season of year), overall aiming to optimize the users wellbeing. Second, the recommendation can be combined with an automatic preparation (e.g. quantity or intensity) of the respective drug or vitamin. The users' reaction (e.g. new blood test results or (verbal) feedback) can be used to adjust and to further personalize the recommendation.

Embodiments of the invention may be provided in the context of EHR cardiovascular prevention. Given a patient's blood sample, it can be predicted (e.g. by KBLRN, which is a framework for end-to-end learning of knowledge base representations from latent, relational, and numerical features) if the patient will suffer from a cardiovascular event in the near future. If this is the case, the doctor can set health goals together with the patient. Embodiments of the invention can then help the patient to keep track of these goals and provides motivational suggestions tailored to the patient's personality type and learnt personal preferences. In particular, today's regular health check can present indications how healthy a person is (e.g. weight). A deeper protein analysis can indicate further risks and correlations to the life-style. Both types of data can, through multi-modal integration of information, provide accurate recommendations. Embodiments of the invention closes the gap of how to provide the recommendations to the patient and at the same time monitors their development based on the actions he/she has to take. As a result, embodiments of invention can increase the prediction quality through AI-based, human-friendly explanations and personalized recommendation adjustments. This in turn decreases the likelihood of the patient experiencing a cardiovascular event in the future.

Embodiments of the invention may be provided in the context of diabetes prevention. For example, the Jazba project is a health checkup service in India, which focuses on Diabetes Type-2 patients. For that purpose, customer's basic information and lifestyle are recorded and in combination with health check-ups, feedback is provided to the customer about their health condition and advice is given concerning what to do to stay healthy. Based on the features such as collected in the Jazba project (e.g. age, height, weight, waist, and/or blood pressure), it can be predicted (e.g. by KBLRN) how likely it is that a patient will develop diabetes. If the risk is too high, the doctor can set health goals together with the patient. Embodiments of the invention can then help the patient to keep track of these goals and provide motivational suggestions tailored to the patient's personality type and learnt personal preferences. Because embodiments of the invention adjust to the user's personality and provides recommendations suitable for the user's personality and needs, the embodiments will decrease the likelihood of the patient developing diabetes.

Embodiments of the invention may be provided in the context of after-care treatment. Surgeons may be interested in providing patient in after-care with a virtual assistant that encourages them to follow the recommended routines to optimally recover from their treatment. This would greatly reduce the number of times patients meet with doctors or have to return to hospitals. Embodiments of the invention may be suitable in order to effectively encourage the patients.

Embodiments of the invention may be provided in the context of machine learning support in job centers. A project such as EcoKnow (cf. https://ecoknow.org) may aim to provide explainability for machine learning predictions in a job center. Embodiments of the invention may enrich the way explanations are displayed by adjusting to the different type of people, e.g. case workers or the category to which a job seeker was assigned (e.g., in Denmark there are 12 categories that people are assigned to at the first job center meeting depending on their personal history and future goals). The explanations may be personalized for individual caseworkers.

Furthermore, at least one embodiment of the invention may have one or more of the following features and characteristics:
The recommendation predictions provided by an individual personalized machine learning model are adapted to the user, his personality type, as well as his mood at the present time. The recommendation predictions as guideline information may comprise information with regard to the suggested activity/action as well as how to best motivate the user.
The data of all users may be utilized for training any model by reweighing the feedback information data of each user depending on their personality type, mood at the time of the feedback collection and goal success. This may be performed by a collaborative personality-based feedback harmonizer.
When updating the personality-typed machine learning models and individual personalized machine learning models, the feedback information that has been collected across different models is biased. Instead of applying collaborative filtering, which throws away some data, the data is reweighed and the bias in the collected feedback information data can be corrected by using counterfactual learning.

According to an embodiment of the invention, the following steps may be performed in order to provide a digital personal assistant method or system to be prepared for a new domain:
Provide a set of goals appropriate for the type of a digital personal assistant to be built.
Provide a set of strategies to achieve the goals and appropriate individual actions.
Train an initial emotional state recognizer.
Train an initial model for each personality type using some initial data repository.

Further, the following steps may be performed by the digital personal assistant method or system for each new user:
Assign one personality type to the user
Give them the initial model that was trained for this personality type
Collect implicit and explicit user feedback, weigh the feedback according to the emotional state recognizer and collect the data in a log of all users

Finally, to update the models of the digital personal assistant system, it may be provided that for all collected data across all personality types a collaborative personality-based feedback harmonizer is used to reweigh the data for further training of individual personalized machine learning models as well as the initial personality-typed machine learning models.

At least one embodiment of the invention may have one or more of the following features and advantages:
Considering the fact that different people need different model outputs based on their personality. Furthermore, while determining an initial personality type is helpful to get a user started with an initially suited model, personalities are rich and complex and as such a model should learn to adapt to a specific user by considering user feedback and their emotional state at the time this feedback was given. At the same time the obtained user feedback is stored in a shared database. Both personal models and personality-typed models can be updated by reweighing the stored data depending on the user's personality and the model to be updated.
Providing a reweighing and de-biasing counterfactual learning approach that ensures that all data can be used, thus leading to higher data efficiency.
Following a user as the user attempt to change his behavior and offer him suggestions to achieve his goal.
Suggesting individual and personalized models for a person. Further, the exchanged training data is weighted based on a person similarity and based on the crucial aspect of the persons' personality. Moreover, a mechanism is provided how to adapt the prediction to the user, e.g., how to present or transform it depending on the mood of the user.

There are several ways how to design and further develop the teaching of the present invention in an advantageous way. To this end, it is to be referred to the patent claims subordinate to patent claim 1 on the one hand and to the following explanation of further embodiments of the invention by way of example, illustrated by the figure on the other hand. In connection with the explanation of the further embodiments of the invention by the aid of the figure, generally further embodiments and further developments of the teaching will be explained.

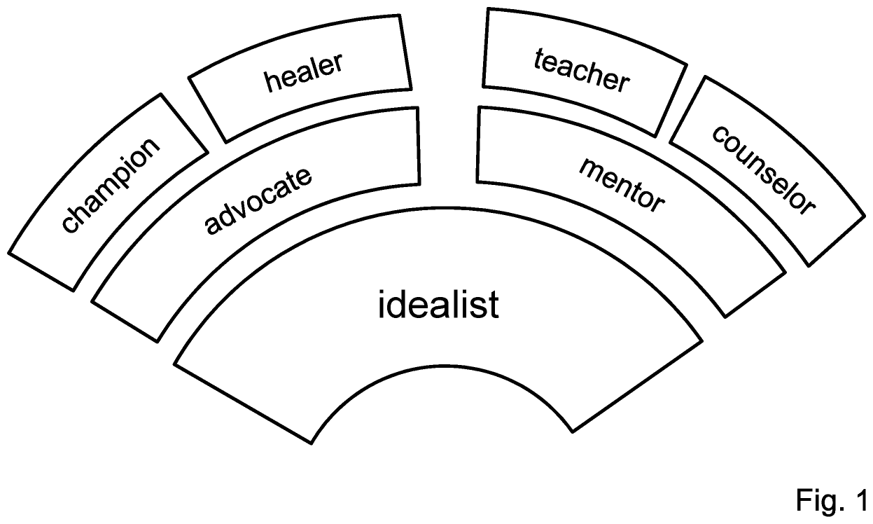

FIG. 1 shows an excerpt of the psychological types (personalities) by Myers-Briggs. The Myers-Briggs types classify people into different groups based on their psychological preferences, i.e., how people make decisions and perceive their periphery. The excerpt of FIG. 1 depicts that different personality types have a common base and share certain properties with other personalities. For more details, it is referred to https://www.myersbriggs.org/my-mbti-personality-type/mbti-basics/home.htm?bhcp=1.

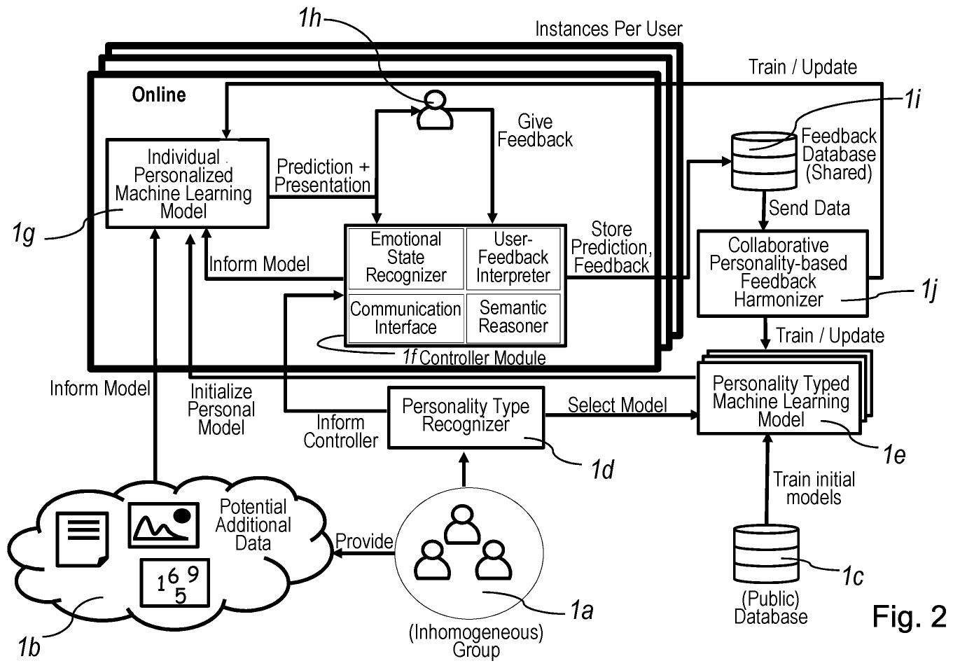

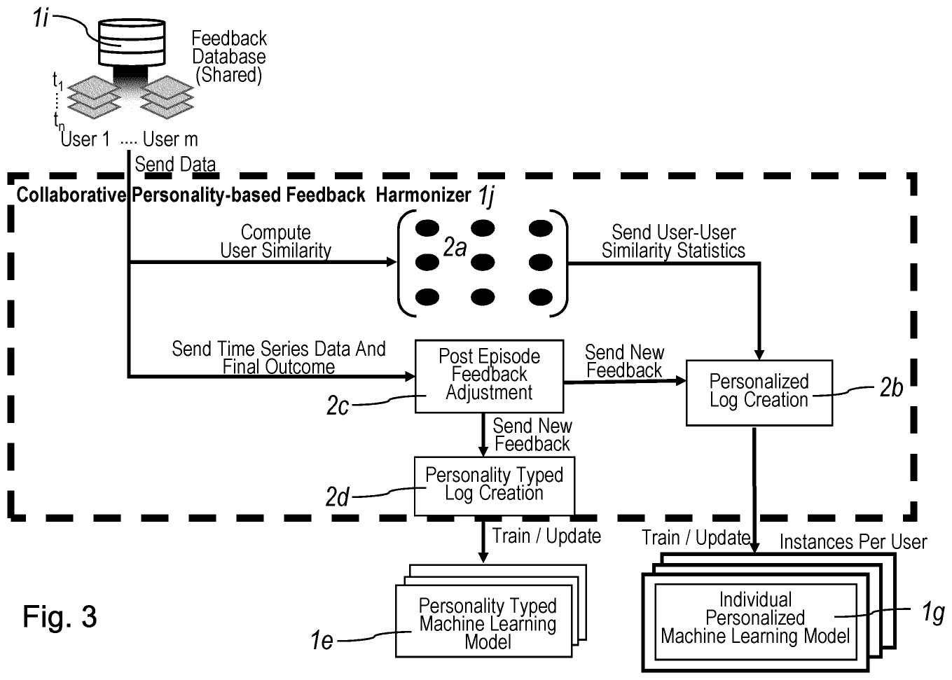

FIG. 2 shows a schematic diagram illustrating a workflow for a method according to an embodiment of the present invention. Given a user (1a), as well as potential additional data from the users (1b), the first step is to classify the user's personality type (1d). Based on the recognized personality type, the corresponding personality-typed machine learning model is selected (1e), from a pool of personality-typed models (one for each type) that were trained based on previously collected data (1c). The selected model is used to initialize this user's personal model (1g) that will be further adapted to the user. Every time the personalized model (1g) offers a recommendation prediction to the user, this prediction is informed by the controller module (1f), which in turn considers the recognized personality type of the user. The controller module may consist of a communication interface, a semantic reasoner, an emotional state recognizer and a user-feedback interpreter, which can influence the model decisions and interact with the user. Once a prediction is presented to the user (1h), the user can give implicit and/or explicit feedback. After the emotional state recognizer and the user-feedback interpreter have processed the feedback, it is stored, together with the model prediction, in a feedback database (1i), which is shared across all users. Periodically, the data collected by the shared feedback database is used to update all personality-typed machine learning models, as well all users' personalized models. Depending on which type of model is updated, the feedback is pre-processed differently by the collaborative personality-based feedback harmonizer (1j). In this context, the different information are exchanged between and combined across users and weighted depending on the user similarity but also the (emotional) state of the user and the personality type. For details of this module, it is referred to FIG. 3.

FIG. 3 shows is a schematic diagram illustrating a collaborative personality-based feedback harmonizer for a method according to an embodiment of the present invention. With the shared feedback database (1i), either an individual personalized model (1g; path top & right) or personality-typed model (1e; path bottom & left) can be updated. For both cases, if the final outcome of a user's logged feedback is known (e.g. whether they reached their goal or not), all feedback data points of this user are readjusted by this final outcome in the Post Episode Feedback Adjustment (2c). For the individual personalized model, a user similarity matrix (2a) is computed which indicates how similar each user is to all other users. Based on this similarity score, the feedback database is reweighed to create a personalized log for a specific user (2b). This personalized log is then used to update the model for this specific user. For the personality-typed models, the logged data is reweighed depending on the personality type mismatch between the personality assigned to the user and the personality type of the model that is to be updated (2d). This personality-typed log is then used to update the model of a specific personality type.

According to embodiments of the invention, in particular as illustrated by FIG. 2 and FIG. 3, a corresponding procedure may be implemented as follows:

In certain contexts (such as gym, smart-home, virtual reality, shopping and/or health), it can be helpful to have model outputs that are personalized to a user. For example, imagine a gym user who wants the most optimal routine. This optimal routine depends not just on their fitness level, but also on their current mood as well as their personality type, which can inform a prediction model on how to best motivate a user. The user will be given an initial model suited for their personality and then the model will learn a user's particular personality over time by observing implicit and explicit rewards and employing reinforcement learning. In order to provide the user with an initial model suited for their personality, it is defined a set of personality types. This set of types could be defined specifically for the task at hand by experts or we could employ a standardized personality type profiler, such as the Myers-Briggs type indicator which can group a person into one of 16 personality types after the person answers a couple of questions. Models are adjusted not just by the feedback given from the user using this model but it will also use feedback given by other users. This is done by pivoting each user off their assigned personality type and feedback data is reweighted based upon the difference in personality type. Respecting different personality types, how they relate to each other and how a machine learning system can process the different aspects that arise from this, is the key idea of this invention.

For a given task, it is assumed that there exists a test, e.g. a set of questions that are answered by the user, with which a personality type can be assigned to a user. We assume this test can give a probability distribution p over personality types gᵢ for a person uⱼ, i.e. μ(g|uⱼ) is a probability vector of the same length as the number of distinct personality types. Once a personality type pⱼ is determined for a user,

$$i . e . via {p}_{j} = {arg max}_{g} μ ( g \vert {u}_{j} ) ,$$

they are given a model trained specifically for this personality type to start out with. Over time, the model will further adapt to the specific user.

Given a user uⱼ, the corresponding personality type pⱼ=gᵢ and profile xⱼ (containing such information as demographic data, problem-dependent data, as well as the personality type), the users individual model π^{u}^{ⱼ} is initialized by setting it to some global model for the user's personality type π^{g}^{ᵢ} (see FIG. 2, 1a-e). The model defines a probability distribution over all possible actions Y(xⱼ) that it can suggest (e.g. the model could be a neural network). Actions can be pre-defined depending on the goal and set by experts but can also be modified and enhanced during the application life-cycle. For example, for a model that assists a person during their gym routine, an action could be to increase the speed of the treadmill. An action could also be to do nothing for the current time slot. Furthermore, actions can be multi-dimensional and next to choosing an activity it could also output how this activity is presented to the user, i.e. in which manner the activity should be presented in order for the user to feel the most motivated about it. At every pre-determined time step, e.g. every minute or hour, an action y is drawn from the probability distribution. The probability of drawing this sample at time step t is denoted as π^{u}^{ⱼ} (yₜ|xⱼ).

Each time an action is selected and shown to the user, implicit and explicit feedback is collected (see FIG. 2, 1f-h). Implicit feedback can for example be collected by measuring the heart rate change after the treadmill speed has been increased. Explicit feedback can be collected by outright asking the user to provide feedback on whether or not they liked the change in speed. Furthermore, to allow the user to have more control, they may also provide more fine-grained feedback such as that the suggested workout time was too long or by selecting or providing a suggestion that they would have preferred. Based on this feedback, the model can adjust to be more uniquely suited for this user.

Further, it is employed an emotional state recognizer (see FIG. 2, 1f) to handle the implicit feedback. This emotional state recognizer can for example register skin temperature, perspiration levels, heart pressure, blood pressure, voice pitch. With this information the system gauges the emotional state of the user, e.g. if the user is stressed. This information can be used in two ways: First, it can inform the predictive model so that the chosen action depends on the emotional state (e.g. if the user is stressed it might be better not to lower the speed of the treadmill again). Second, it can be used to reweigh feedback given by the user (e.g., feedback given while the user is stressed could be deemed more or less valuable).

Feedback, both implicit and explicit, will be converted so that it represents a real valued number S. This feedback is saved in an external database (see FIG. 2 or FIG. 3, 1i) that is shared across users. For each user uⱼ, we record their profile xⱼ, their personality type pⱼ and once they have stopped using the system for their goal, we record the success rate of reaching that goal as Δⱼ. For each user j, and for each time step t, we record the action y_{j,t} (where we abbreviate yₜ when the context of user j is clear) chosen by the model with probability π^{u}^{ⱼ} (yₜ|xⱼ) and the feedback δ_{j,t} (where we abbreviate δₜ when the context of user j is clear).

Periodically, we update both the personalized models and the personality typed models (see FIG. 2, 1e and 1g), using all data that is shared across users (see FIG. 2, 1i). For both cases, if the final outcome Δⱼ at the final time step T of a user's logged feedback is already known, all feedback data points of this user are readjusted by this final outcome in a post episode feedback adjustment via δₜ=γ^{(T-t+1)δt} Δⱼ. This ensures that feedback achieved closer to the final time step T is weighed higher as it has a higher impact to reaching the final outcome Δⱼ than feedback achieved at earlier time steps.

For the personalized model updates (see FIG. 3), a user-user similarity matrix S is first computed, where entry sⱼⱼ′ denotes the similarity between user uⱼ and user uⱼ′. To utilize the data from user uⱼ′ to update the model of user uⱼ, the feedback at a time t is reweighed in the following way: δ_{j′,t}=sⱼⱼ′·δ_{j′,t}. The user similarity metric allows to use and weight training data across users and groups. For example, users that are similar share training data, whereas less similar users only share a little or even negatively. Once the data of all users is reweighed, the resulting personalized log can be used to update model π^{u}^{ⱼ} (see FIG. 3, 2b).

For the personality-typed model updates, a model π^{g}^{ᵢ} for personality type gᵢ can be directly updated using data that originates from users uⱼ that where assigned personality type pⱼ=gᵢ (see FIG. 3, 2d). However, to harness synergies across personality types and to increase the volume of available data, we propose to use data from a user of type pⱼ=gᵢ to update a model for type g_{i′} by rescaling the reward by how likely it would have been to instead ascribe type g_{i′} to the user, i.e. we rescale δ_{j, t}=μ(g_{i′}|uⱼ)·δ_{j,t}, where we recall that μ(g_{i′}|uⱼ) defines the probability of the user belonging to personality type g_{i′}. This idea honours the fact that although we can only choose one personality type when deciding which model to roll out to a user, a user can also have a certain probability to belong to another personality type. It is noted that this step can be done even if a user's personality type pⱼ=g_{i′} by respecting the fact that user uⱼ might not only exhibit characteristics of their assigned personality type (see FIG. 1).

When utilizing data that was collected under one model to update another model, there is an inherent bias in the collected data that should be corrected because if we had a different model, it would have chosen different actions and we would have collected different feedback. To handle this issue during learning, counterfactual estimators, such as Inverse Propensity Scoring (IPS) (cf. Paul R. Rosenbaum and Donald B. Rubin. 1983. The central role of the propensity score in observational studies for causal effects. Biometrika, Vol. 70, No. 1, which is retrievable at http://www.stat.cmu.edu/-ryantibs/journalclub/rosenbaum_1983.pdf) can be used which counter the bias using importance sampling. This is a crucial step as otherwise learning might deteriorate due to the bias in the data. For example, as described in Richa Khandelwal. “Serving Athletes* With Personalized Workout.” 2018, previous work typically uses collaborative filtering, which fully deletes data that does not fit for training a certain model. In contrast, embodiments of the invention provides a reweighing and counterfactual learning approach that ensures that all data can be used, leading to higher data efficiency.

Additionally, embodiments of the invention can give the user the control over their private data, so that the user can decide which type of data the model is allowed to access and which type of data will be allowed to be stored in the external feedback database.

Embodiments of the invention may also be able to provide explainability features to the user so that the user can understand why a certain decision was made at a particular point in time.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A method of providing personalized guideline information for a user in a predetermined domain, wherein a set of personality types is defined for users of said predetermined domain, the method comprising:
determining, by a personality type recognizer, a personality type for a user in order to assign the personality type to said user;
selecting a personality-typed machine learning model from a model pool of personality-typed machine learning models based on the personality type of said user, wherein the selected personality-typed machine learning model is used to initialize an individual personalized machine learning model of said user; and
generating, by the individual personalized machine learning model of said user, a recommendation prediction, wherein the recommendation prediction is presented as a guideline information to said user.

2: The method according to claim 1, wherein the personality-typed machine learning models of the model pool were trained based on previously collected data.

3: The method according to claim 1, wherein a controller module is provided, wherein said controller module is configured to inform the individual personalized machine learning model.

4: The method according to claim 3, wherein every time the individual personalized machine learning model presents a recommendation prediction to said user, the individual personalized machine learning model is informed by the controller module, wherein the controller module in turn considers the recognized personality type of said user.

5: The method according to claim 3, wherein said controller module is configured to interact with said user.

6: The method according to claim 3, wherein the control module is configured to collect feedback information from said user when a recommendation prediction is presented to said user.

7: The method according to claim 3, wherein the controller module includes an emotional state recognizer, wherein the emotional state recognizer is employed to handle implicit feedback information of said user such that an emotional state of said user is determined.

8: The method according to claim 7, wherein the emotional state of said user is considered by the individual personalized learning model of said user, and/or
wherein the emotional state of said user is used to reweigh the feedback information given by said user.

9: The method according to claim 6, wherein the feedback information is logged together with the recommendation prediction in a feedback database, wherein said feedback database is shared across all users of the predetermined domain.

10: The method according to claim 9, wherein, feedback information of users collected by the feedback database is used to update personality-typed machine learning models of the model pool and/or to update users' individual personalized machine learning models.

11: The method according to claim 9, wherein, if a final outcome of a user's logged feedback information is known, all feedback information data points of the user are readjusted by the final outcome in a post episode feedback adjustment.

12: The method according to claim 9, wherein a user similarity matrix is computed, wherein the user similarity matrix indicates how similar each user is to all other users, such that, based on a similarity score, logged feedback information of the feedback database is reweighed to create a personalized log for a specific user, wherein the personalized log is used to update the individual personalized machine learning model of the specific user.

13: The method according to claim 9, wherein, depending on a personality type mismatch between the personality type assigned to a user and a specific personality type of a specific personality-typed machine learning model that is to be updated, logged feedback information of the feedback database is reweighed to create a personality-typed log, wherein the personality-typed log is used to update the specific personality-typed machine learning model having the specific personality type.

14: The method according to claim 1, wherein the predetermined domain includes a gym environment, a smart home environment, a virtual reality environment, a shopping environment and/or a health environment.

15: A system of providing personalized guideline information for a user in a predetermined domain, wherein a set of personality types is defined for users of said predetermined domain, the system being configured to:
determine, by a personality type recognizer, a personality type for a user in order to assign the personality type to said user,
select a personality-typed machine learning model from a model pool of personality-typed machine learning models based on the personality type of said user, wherein the selected personality-typed machine learning model is used to initialize an individual personalized machine learning model of said user, and
generate, by the individual personalized machine learning model of said user, a recommendation prediction, wherein the recommendation prediction is presented as a guideline information to said user.

16: The method according to claim 1, wherein the predetermined domain is in the context of everyday activities.

17: The method according to claim 6, wherein the feedback information comprises implicit and/or explicit feedback information.

18: The method according to claim 10, wherein the feedback information of users is used periodically.
